(19) United States
(12) Patent Application Publication
(10) Pub. No.: US 2009/0306546 A1
(43) Pub. Date: Dec. 10, 2009

(54) Kink-Resistant Guidewire Having Increased Column Strength

(75) Inventor: Tracey Knapp, Lawrenceville, GA (US)

Correspondence Address: Rutan & Tucker, LLP., 611 ANTON BLVD, SUITE 1400, COSTA MESA, CA, 92626, US

(73) Assignee: C.R. Bard Inc., Murray Hill, NJ (US)

(21) Appl. No.: 12/159,608

(22) PCT Filed: Dec. 21, 2006
(86) PCT No.: PCT/US06/48948

§ 371 Date: Dec. 22, 2008

Related U.S. Application Data
(60) Provisional application No. 60/754,539, filed on Dec. 28, 2005.

(51) Int. Cl. A61M 25/09

(52) U.S. Cl. 600/585

(57) ABSTRACT

A guidewire a distal end and a proximal end, and a core wire having an inner core and an outer layer surrounding at least a portion of the inner core, the outer layer being tapered along the distal end of the guidewire, wherein one of the inner core and the outer layer is formed from at least one kink-resistant material and the other of the inner core and the outer layer is formed from at least one high column strength material.

This application claims priority to U.S. Provisional Application No. 60/754,539, filed Dec. 28, 2005, the contents of which are incorporated herein by reference.

Medical guidewires are typically used to facilitate insertion of a medical device into a vessel of the body during a surgical procedure. For example, an elongated guidewire is normally inserted into the urinary tract prior to inserting a ureteroscope. In such a case, the ureteroscope is advanced over the guidewire as it is inserted into the urinary tract with the guidewire providing a path for the ureteroscope to traverse.

Given that the vessel through which a guidewire is passed may comprise various constrictions or obstacles, it is normally desirable for the guidewire to have relatively high column strength. With such column strength, the guidewire can be advanced past the constrictions or obstacles in the vessel from outside the body without buckling. Traditionally, such column strength was provided by manufacturing the guidewire from stainless steel. Although stainless steel is a material that has relatively high column strength, it is also relatively ductile such that it can deform and set in a new orientation. Because of that deformability, stainless steel guidewires can kink during use. In such a case, a sharp bend may be formed along the length of the guidewire that creates an impediment to advancing a medical device over the guidewire.

Because of the propensity for stainless steel guidewires to kink, shape-memory materials have become popular for the construction of medical guidewires. An example of such materials are nickel-titanium alloys, commonly referred to as nitinol. Nitinol guidewires can be aggressively bent or contorted without kinking.

Although nitinol guidewires have desirable kink resistance, they do not possess the column strength of stainless steel guidewires. However, it may be difficult or impossible to advance the guidewire past the constriction or obstruction given that the guidewire is likely to buckle and coil in such a circumstance. In view of the above, it would be desirable to have a guidewire that is kink resistant and that has relatively high column strength.

### SUMMARY

According to various embodiments, there is provided a guidewire comprising a proximal end and a distal end, and a core wire having an inner core and an outer layer that surrounds at least a portion of the inner core, the outer layer being tapered along the distal end of the guidewire, wherein one of the inner core and the outer layer is formed from at least one kink-resistant material, and the other of the inner core and the outer layer is formed from at least one high column strength material.

### BRIEF DESCRIPTION OF THE DRAWINGS

The disclosed guidewires can be better understood with reference to the following drawings. The components in the drawings are not necessarily to scale.

FIG. 1 is a side view of a first embodiment of a kink-resistant guidewire having increased column strength.

FIG. 2 is a cross-sectional view of the guidewire of FIG. 1 taken along line 2-2.

FIG. 3 is a side view of a first embodiment of a distal end for the guidewire of FIGS. 1 and 2.

FIG. 4 is a side view of a second embodiment of a distal end for the guidewire of FIGS. 1 and 2.

FIG. 5 is a side view of a third embodiment of a distal end for the guidewire of FIGS. 1 and 2.

FIG. 6 is a side view of a fourth embodiment of a distal end for the guidewire of FIGS. 1 and 2.

FIG. 7 is a side view of a fifth embodiment of a distal end for the guidewire of FIGS. 1 and 2.

FIG. 8 is a side view of a sixth embodiment of a distal end for the guidewire of FIGS. 1 and 2.

FIG. 9 is a side view of a second embodiment of a kink-resistant guidewire having increased column strength.

FIG. 10 is a cross-sectional view of the guidewire of FIG. 9 taken along line 10-10.

### DESCRIPTION

As is described in the foregoing, stainless steel guidewires have desirable column strength but tend to kink, while shape-memory material wires have desirable kink resistance but have relatively poor column strength. A guidewire having both desirable kink resistance and column strength can be obtained when the guidewire comprises both a shape-memory material and a high column strength material. In some embodiments, the guidewire includes a core wire that has a core of flexible, shape-memory material and an outer layer of high column strength material.

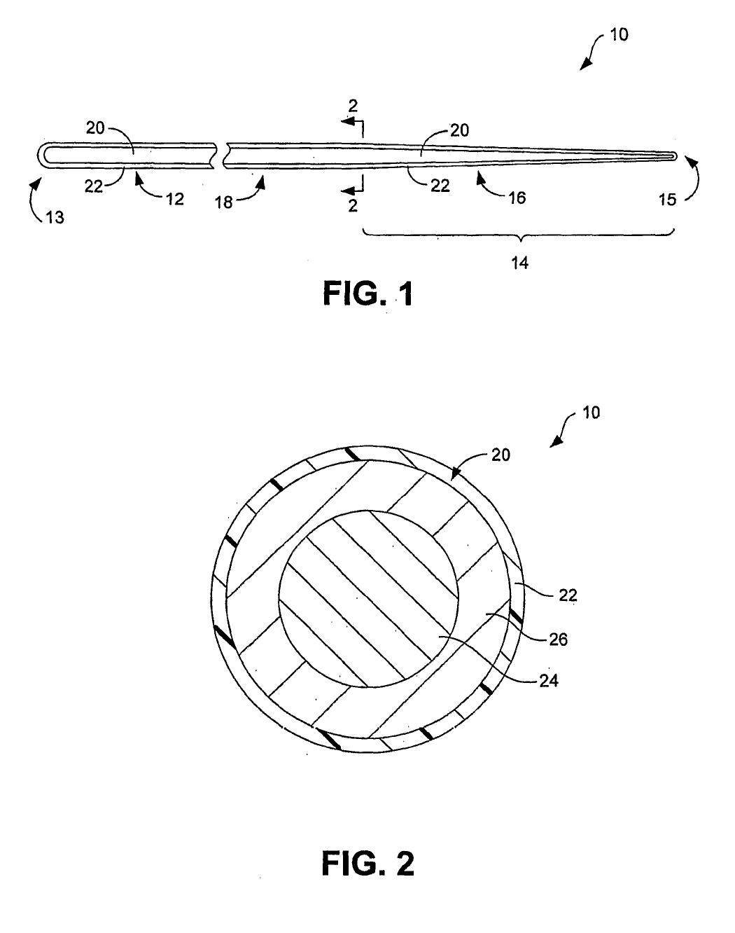

Referring now to the drawings in which like reference numerals identify corresponding components, FIGS. 1 and 2 illustrate an embodiment of a first guidewire 10, which can be used to introduce other devices into a patient vessel such as a urinary tract or a blood vessel. As is indicated in FIG. 1, the guidewire 10 includes a proximal end 12 and a distal end 14, which is adapted for insertion into a patient. The distal end 14 includes a taper 16 that facilitates insertion and provides added flexibility that reduces the potential for injury to the patient. Proximal of the distal end 14 is a shaft 18 that has a uniform outer diameter throughout its length. By way of example, the shaft 18 ranges from about 50 centimeters (cm) to about 500 cm long. According to another embodiment, the shaft 18 ranges from about 70 cm to about 450 cm long, such as from about 125 cm to about 180 cm long. For example, the shaft 18 can have an outer diameter ranging from about 0.005 inches (in.) to about 0.050 in., or from about 0.025 in. to about 0.038 in. The distal end 14 typically ranges from about 3 cm to about 25 cm long. According to another embodiment, the distal end 14 ranges from about 5 cm to about 20 cm long.

As is further indicated in FIG. 1, the guidewire 10 includes a core wire 20 that is surrounded by an outer layer or jacket 22 of material, such as a thermoplastic (e.g., polyurethane or nylon). In addition to the jacket 22, the guidewire 10 can, optionally, be coated with a lubricious, hydrophilic or hydrophobic coating (not illustrated). Notably, in accordance with various embodiments, the guidewire 10 of FIGS. 1 and 2 does not include an outer coiled wire. The absence of such a coiled wire provides the advantages of increased torqueability and simplified, and therefore less expensive, manufacturing.

Referring to FIG. 2, the core wire 20, and the jacket 22 that surrounds it, has a generally round (e.g., circular) cross-section. The core wire 20 may therefore be referred to as a round wire. However, in accordance with various embodiments, the core wire 20 and jacket 22 can independently be of other configurations, such as an oval configuration. As is indicated in FIG. 2, the core wire 20 is a composite wire that includes an inner core 24 composed of a first material and a coaxial outer layer 26 composed of a second material. In accordance with various embodiments, core 24 and coaxial outer layer 26 can each independently be composed of multiple materials. The multiple materials can be combined into a single composite material, and can be provided as multiple layers of materials. In some embodiments, the core 24 is composed of at least one kink-resistant material and the outer layer 26 is composed of at least one high column strength material. In other embodiments, the core 24 is composed of the at least one high column strength material and the outer layer 26 is composed of the at least one high kink-resistant material.

The “kink-resistant material” can comprise, for example, a flexible, shape-memory material, such as a nickel and titanium alloy (commonly referred to as “nitinol”), or another material having similar mechanical properties. Suitable non-limiting examples of such materials include one or more of titanium-palladium-nickel, nickel-titanium-copper, gold-cadmium, iron-zinc-copper-aluminum, titanium-niobium-aluminum, uranium-niobium, hafnium-titanium-nickel, iron-manganese-silicon, nickel-titanium, nickel-iron-zinc-aluminum, copper-aluminum-iron, titanium-niobium, zirconium-copper-zinc, and nickel-zirconium-titanium alloys. The high column strength material can comprise, for example, stainless steel or another high-strength, biocompatible metal.

The relative sizes (e.g., diameters) of the core 24 and outer layer 26 can be selected to provide the desired amount of kink-resistance and column strength. Assuming core 24 comprises the flexible, shape-memory material and the outer layer 26 comprises the high column strength material, the size of the core relative to the outer layer can be increased to provide greater kink-resistance, or decreased to provide greater column strength. In accordance with various embodiments, with the core 24 comprising the inner diameter of core wire 20, the ratio of the inner diameter of the core wire to the total diameter of the core wire can range from about 1% to about 99% of the total diameter of the core wire.

The outer layer 26 can be provided on the core 24 using various different methods. In some embodiments, the core 24 and the outer layer 26 are drawn together such that the core wire 20 is formed in a one-step process. In other embodiments, the outer layer 26 is formed as an independent tube that is passed over the core 24 and secured thereto. In such a case, the outer layer 26 can be secured to the core 24 at discrete locations along the core or along its entire length using any one of several bonding methods including welding, soldering, brazing, applying adhesive, or applying pressure.

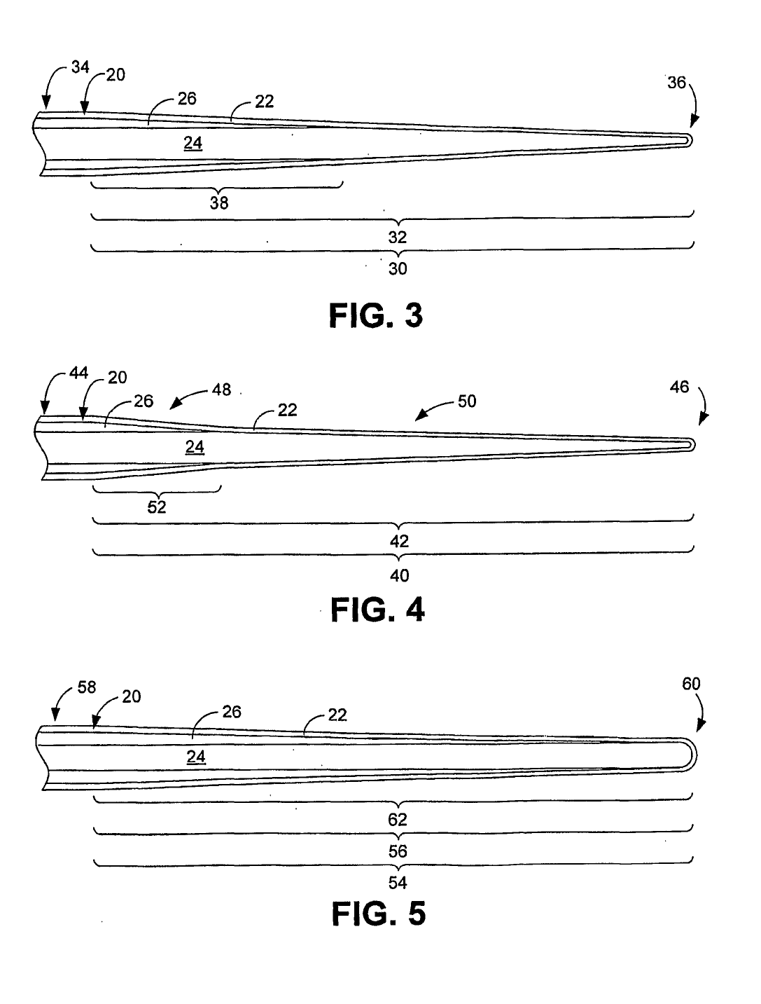

FIGS. 3-8 illustrate example distal ends for the guidewire 10. As is mentioned above, and in accordance with various embodiments, it can be suitable to provide a guidewire having a flexible distal end so as to reduce the possibility of harming the patient. In particular, it might be suitable to provide a very flexible distal end that will yield when urged against the wall of a patient vessel or cavity so that perforation or tearing of the vessel or cavity is avoided, or at least minimized, as the guidewire 10 is advanced. Such flexibility can be achieved when, for example, the core 24 of the wire 20 comprises the flexible, shape-memory material and the outer layer 26 comprises the high column strength material, given that the amount of material comprising the outer layer along the taper 16 of the distal end 14 is reduced. Such a configuration can allow the mechanical properties of the core material to dominate. It is assumed that the core 24 is composed of the flexible, shape-memory material and the outer layer 26 comprises the high column strength material for the discussion of FIGS. 3-8 that follows.

Beginning with FIG. 3, illustrated is a first distal end 30 that can be used for construction of the guidewire 10 of FIG. 1. In this embodiment, the distal end 30 of core wire 20 comprises a uniform taper 32 that extends along the entire length of the distal end 30, from the shaft 34 to the distal tip 36. By way of example, the uniform taper 32 is formed using a grinding process in which both material of the core 24 and the outer layer 26 are removed from the distal end 30. With the uniform taper 32, a transition area 38 is defined in which the amount of high column strength material (e.g., stainless steel) is gradually reduced such that the flexibility of the distal end 30 increases along that area. In addition to providing this gradual transition from relative stiffness to relative flexibility, the uniform taper 32 can further simplify the manufacturing process given that only one grinding process with a single grinding bit is necessary.

Referring next to FIG. 4, illustrated is a second distal end 40. In this embodiment, the core wire 20 comprises a non-uniform taper 42 that extends along the entire length of the distal end 40, from the shaft 44 to the distal tip 46. As indicated in FIG. 4, the taper 42 affects both the core 24 and the outer layer 26. By way of example, the non-uniform taper 42 comprises a first taper 48 adjacent the shaft 44 that transitions into a second taper 50 that extends to the distal tip 46. In such a case, the first taper 48 is greater (i.e., less gradual) than the second taper 50 such that the transition area 52 in which the high column strength is reduced is smaller than that of the distal end 30 shown in FIG. 3. This results in the amount of high column strength material (i.e., the outer layer 26) in the distal end 40 being reduced relative to the embodiment shown in FIG. 3, thereby increasing the flexibility of the distal end 40.

With reference to FIG. 5, illustrated is a third distal end 54. In this embodiment, only the outer layer 26 is tapered such that the core 24 is substantially uniform in diameter throughout its length. This result can be achieved by providing either a uniform or non-uniform taper 56 that reduces the outer layer 26 along the entire length of the distal end 54, from the shaft 58 to the distal tip 60 of the distal end 54. In such an arrangement, the distal end 54 is relatively stiff, but gradually reduces in stiffness (i.e., increases in flexibility) as the taper 56 is traversed to the distal tip 60. The outer layer 26, and therefore the amount of high column strength material, is decreased along an elongated transition area 62 that extends along substantially the entire distal end 54 such that the outer layer extends along substantially the entire core wire 20. Due to the taper, however, very little high column strength material, if any, remains at the distal tip 60 of the distal end 54.

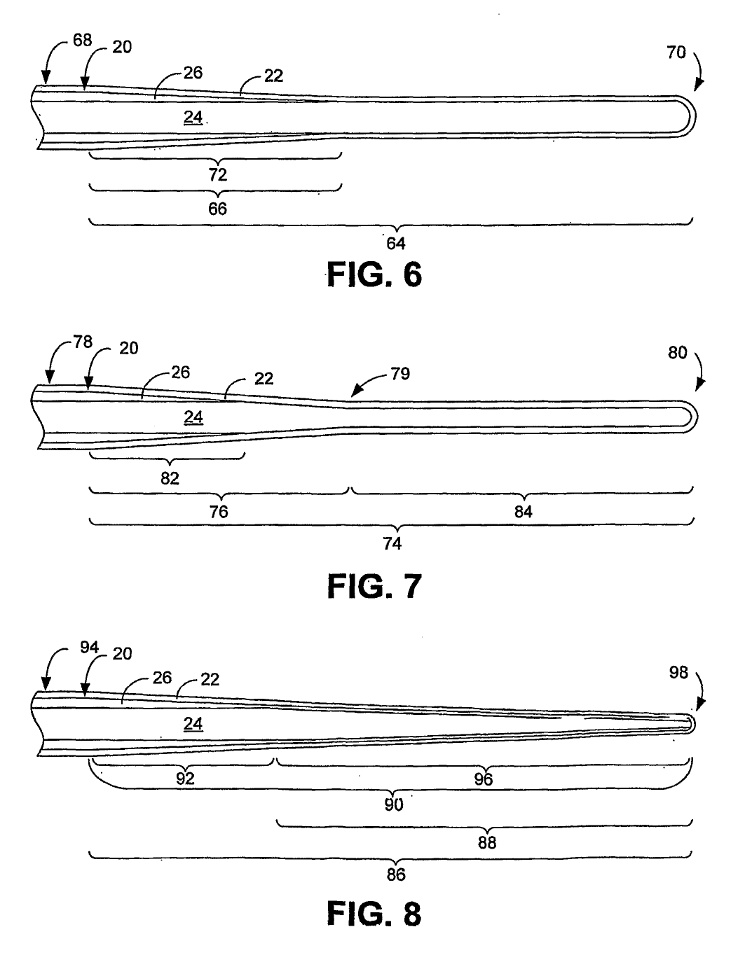

FIG. 6 illustrates a fourth distal end 64 for the guidewire 10. As with the embodiment of FIG. 5, only the outer layer 26 is tapered. This result is achieved by providing a uniform or non-uniform taper 66 that extends from the shaft 68. The taper 66 does not, however, extend along the entire length of the distal end 64 to the distal tip 70. The taper 66 can be relatively short to define a relatively short transition area 72 for the high column strength material or relatively long to define a relatively long transition area. Given that a substantial portion of the distal end 64 comprises only the core 24, and therefore the flexible, shape-memory material, the distal end 64 is more flexible that the distal end 54 of FIG. 5.

Referring now to FIG. 7, illustrated is a fifth distal end 74. In this embodiment, both the outer layer 26 and the core 24 are tapered by a taper 76 that extends from the shaft 78 to a location 79 prior to the distal tip 80 of the distal end 74. This arrangement results in a transition area 82 for the high column strength material that is shorter than the total length of the taper 76. Given that the taper 76 extends beyond the transition area 82 but short of the distal tip 80 of the distal end, the core 24 comprises a uniform, reduced-diameter portion 84 that extends from the taper to the distal tip. Such a portion 84 can be formed, for example, through a barrel grinding process.

FIG. 8 illustrates a sixth distal end 86 that comprises a core 24 having a uniform taper 88 and an outer layer 26 having a non-uniform taper 90. In this embodiment, the non-uniform taper 90 comprises a first portion 92 that extends from the shaft 94 to the core taper 88 and a second portion 96 that extends along the core taper 88 to the distal tip 98 of the distal end 86. With such an arrangement, the amount of high column strength material is reduced along the first portion 92, but then remains constant along the second portion 96. The high column strength material therefore extends along substantially the entire core wire 20, but is reduced in mass along the second portion 96 to an extent at which the flexible, shape-memory material of the core 24 dominates the properties of the wire adjacent the distal tip 98.

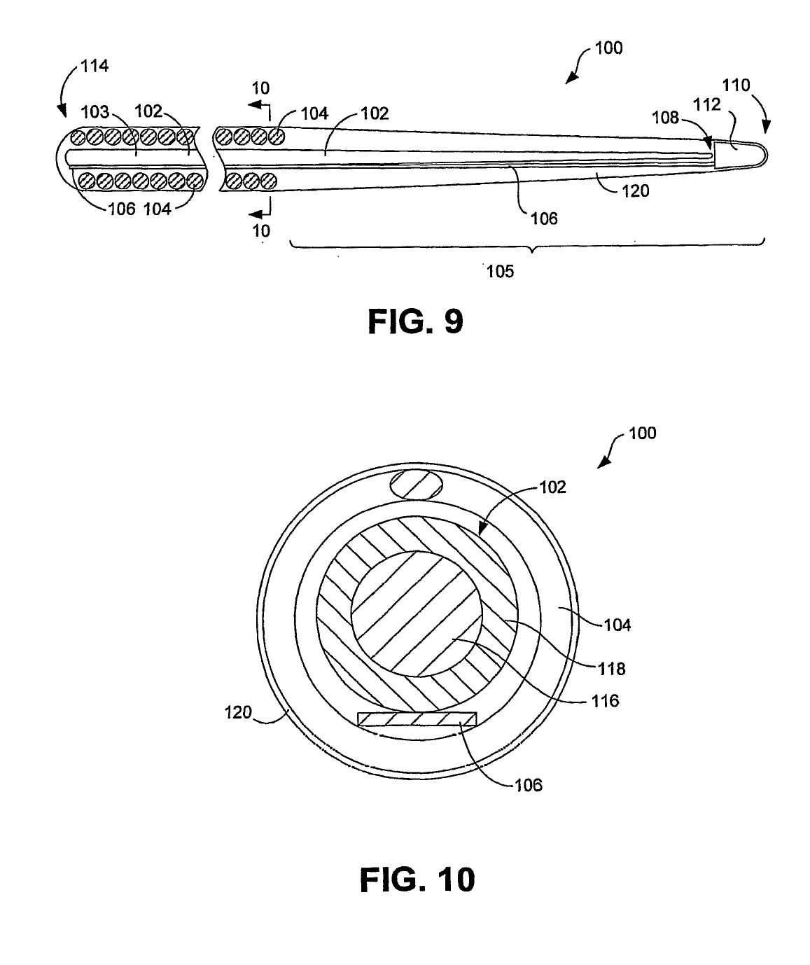

FIGS. 9 and 10 illustrate an embodiment of a second guidewire 100. In this embodiment, the guidewire 100 includes a core wire 102, a coiled wire 104, and a safety wire 106 that is positioned between the core wire and the coiled wire. The core wire 102 can have a configuration similar to that described above in relation to FIGS. 1 and 2. As is indicated in FIG. 9, the coiled wire 104 surrounds the core wire 102 along the shaft 103 of the core wire. The distal end 105 of the guidewire wire 102 can have a configuration similar to any of those described in relation to FIGS. 3-8. The distal tip 108 of the core wire 102 is tapered and extends toward the distal tip 110 of the guidewire 100.

The safety wire 106 extends to a weld 112 provided within the distal tip 110 of the guidewire 100, and can be secured to the weld using any appropriate bonding method, including, for example, welding, soldering, brazing, or using adhesive. The coiled wire 104 and the safety wire 106 can be secured together at discrete locations along the length of the safety wire, or along the entirety of the length of the coiled wire 104. Moreover, each of the core wire 102, coiled wire 104, and safety wire 106 can be secured together at the proximal end 114 of the guidewire 100.

As is apparent from FIG. 10 the core wire 102 is a composite wire and therefore comprises a core 116 and a coaxial outer layer 118. As with the guidewire 10 of FIGS. 1 and 2, the core 116 can comprise a flexible, shape-memory material (e.g., nitinol) and the outer layer 118 can comprise a high column strength material (e.g., stainless steel), or vice versa. The core wire 102, coiled wire 104, and the safety wire 106 can be coated with a layer 120 of polymeric material, lubricious material, hydrophilic material, and/or other material.

In some embodiments, the coiled wire 104 and the safety wire 106 are composed of the high column strength material. In embodiments in which the outer layer 118 of the core wire. 102 comprises the same material as the coiled wire 104 and the safety wire 106 (e.g., stainless steel), those components can be welded together at the proximal end 114. Alternatively, other bonding methods described herein can be used to secure the core wire 102, coiled wire 104, and safety wire 106 together at the proximal end 114.

From the foregoing, it can be appreciated that guidewires having both desirable kink-resistance and column strength can be achieved using composite wires including both flexible, shape-memory material and high column strength material. Furthermore, a desired amount of tip flexibility can be achieved using various different distal end configurations.

For the purposes of this specification and appended claims, unless otherwise indicated, all numbers used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a guidewire” includes two or more guidewires.

Other various embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A guidewire comprising:
a proximal end and a distal end; and
a core wire having an inner core and an outer layer that surrounds at least a portion of the inner core, the outer layer being tapered along the distal end of the guidewire;
wherein one of the inner core and the outer layer is formed from at least one kink-resistant material, and the other of the inner core and the outer layer is formed from at least one high column strength material.

2. The guidewire of claim 1, wherein the distal end of the guidewire does not contain coiled wire.

3. The guidewire of claim 1, wherein the inner core is formed from at least one kink-resistant material and the outer layer is formed from at least one high column strength material.

4. The guidewire of claim 3, wherein the inner core comprises a shape-memory material.

5. The guidewire of claim 4, wherein the shape-memory material comprises a nitinol material.

6. The guidewire of claim 3, wherein the outer layer comprises stainless steel.

7. The guidewire of claim 1, wherein the inner core is tapered.

8. The guidewire of claim 7, wherein the inner core comprises a non-tapered proximal end and a tapered distal end.

9. The guidewire of claim 1, wherein the inner core is untapered.

10. The guidewire of claim 1, wherein the outer layer is tapered along a transition area of the distal end and wherein the transition area ends before a tip of the distal end.

11. The guidewire of claim 1, wherein the outer layer is tapered along a transition area of the distal end and wherein the transition area substantially extends to a tip of the distal end.

12. The guidewire of claim 1, further comprising a jacket surrounding at least a portion of the core wire.

13. The guidewire of claim 1, further comprising a coiled wire that surrounds at least a portion of the core wire.

14. The guidewire of claim 13, further comprising a jacket surrounding at least a portion of the core wire and the coiled wire.

15. The guidewire of claim 1, wherein the distal end is uniformity tapered.

16. The guidewire of claim 1, wherein the distal end is non-uniformily tapered.

17. A guidewire comprising:
a proximal end and a distal end;
a core wire including an inner core formed of a shape-memory material and an outer layer that surrounds at least a portion of the inner core, the outer layer being formed of a high column strength material, the outer layer being tapered along the distal end of the guidewire; and
a jacket that surrounds the core wire.

18. The guidewire of claim 1, wherein the distal end of the guidewire does not contain coiled wire.

19. The guidewire of claim 17, wherein the inner core is formed from a nitinol material.

20. The guidewire of claim 17, wherein the outer layer is formed from stainless steel.
